(19) United States
(12) Patent Application Publication
(10) Pub. No.: US 2025/0049870 A1
(43) Pub. Date: Feb. 13, 2025

(54) PHARMACEUTICAL COMPOSITION FOR HEALING ABDOMINAL PAIN AND FLATULENCE

(71) Applicant: TENG SUH LEE, Kuala Lumpur (MY)

(72) Inventor: TENG SUH LEE, Kuala Lumpur (MY)

(21) Appl. No.: 18/655,307

(22) Filed: May 5, 2024

(30) Foreign Application Priority Data
May 30, 2023 (MY) PI2023003199

(51) Int. Cl. A61K 36/076; A61K 9/00; A61K 9/14; A61K 36/232; A61K 36/235; A61K 36/258; A61K 36/284; A61K 36/36; A61K 36/481; A61K 36/484; A61K 36/53; A61K 36/534; A61K 36/54; A61K 36/58; A61K 36/65; A61K 36/66; A61K 36/752; A61K 36/754; A61K 36/8888; A61K 36/8905; A61K 36/8945; A61K 36/899; A61K 36/8998; A61K 36/906; A61K 36/9062; A61K 36/9064; A61K 36/9066; A61K 36/9068

(52) U.S. Cl. CPC A61K 36/076; A61K 9/0014; A61K 9/0053; A61K 9/009; A61K 9/14; A61K 36/232; A61K 36/235; A61K 36/258; A61K 36/284; A61K 36/36; A61K 36/481; A61K 36/484; A61K 36/53; A61K 36/534; A61K 36/54; A61K 36/58; A61K 36/65; A61K 36/66; A61K 36/752; A61K 36/754; A61K 36/8888; A61K 36/8905; A61K 36/8945; A61K 36/899; A61K 36/8998; A61K 36/906; A61K 36/9062; A61K 36/9064; A61K 36/9066; A61K 36/9068

(57) ABSTRACT

The present invention relates to a pharmaceutical composition for healing abdominal pain and flatulence. The pharmaceutical composition comprises a combination of herbal components, which serve as active ingredients. The herbal components in the composition are Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita.

### FIELD OF THE INVENTION

The present invention relates to a pharmaceutical composition. More particularly, the present invention relates to a pharmaceutical composition for healing abdominal pain and flatulence.

### BACKGROUND OF INVENTION

Flatulence and abdominal pain are common clinical signs that often accompany various gastrointestinal disorders, such as acute and chronic pancreatitis, gastrointestinal spasm, incomplete intestinal obstruction, tuberculous peritonitis, abdominal allergic purpura, irritable bowel syndrome (IBS), and dyspepsia abdominal pain. These symptoms, characterized by an increase in gas production, can be indicative of underlying inflammation, obstruction, or functional abnormalities within the gastrointestinal tract. While conventional medical treatments utilizing modern pharmaceutical drugs have certain limitations in terms of providing effective relief and may be associated with unwanted side effects, there has been an increasing curiosity in exploring complementary and alternative medicines (CAM) as an alternative approach. For instance, common conventional drugs used for abdominal pain relief include nonsteroidal anti-inflammatory drugs (NSAIDs) like ibuprofen, antispasmodics such as dicyclomine, and proton pump inhibitors (PPIs) like omeprazole. Despite their widespread use, these medications may not always offer satisfactory results and can have adverse effects, prompting individuals to seek alternative treatment options like CAM. CAM treatments encompass a range of modalities, including dietary supplements, Traditional Chinese Medicine (TCM), acupuncture, moxibustion, and botanicals, which have shown promise in improving abdominal symptoms and enhancing the overall quality of life.

With a history spanning five thousand years, Traditional Chinese Medicine (TCM) has been deeply ingrained in Chinese culture and has been employed as a therapeutic practice for various ailments, including abdominal pain and flatulence. One particularly notable TCM remedy is the Tong-Xie-Yao-Fang (TXYF) decoction, which dates back to the 16th century and is documented in the TCM classic ‘Jingyue's Complete Works.’ This decoction has traditionally been used to address abdominal pain with diarrhea caused by liver-spleen disharmony. Building upon the TXYF decoction, a modified version known as Shun-Qi-Tong-Xie (SQTX) Granule has been specifically developed to target abdominal pain, distention, and diarrhea in individuals with irritable bowel syndrome, IBS-D (liver-spleen disharmony). The formulation of SQTX Granule incorporates several key Chinese herbs, including Baishao (Paeonia lactiflora Pall.), Baizhu (Atractylodes macrocephala Koidz.), Xiebai (Allium macrostemon Bunge.), Cheqianzi (Hegba Plantaginis Asiaticae), Foshou (Citrus medica L. var. sarcodactylis Swingle), and Boheyou (Oleum Mentha haplocalyx Briq.) [Wang et. al., Trials 2014, 15:273]. Furthermore, another Chinese patent publication (CN111773355A, issued on 16 Oct. 2020) discloses a Traditional Chinese Medicine (TCM) composition specifically for treating stomach illnesses, including gastric issues. This composition contains various ingredients such as Atractylodes macrocephala, ginseng, licorice, dried ginger, psoralen, white cardamom, tangerine peel, herb, pinellia, poria cocos, evodia, divine comedy, Magnolia officinalis, amomum, and betel nut.

Building upon the rich history and effectiveness of Traditional Chinese Medicine (TCM), these complementary and alternative medicines (CAM) approaches offer promising alternative options for managing abdominal pain and flatulence. By incorporating key Chinese herbs and utilizing TCM principles, these treatments have shown the potential in providing relief and improving the quality of life for individuals experiencing gastrointestinal symptoms. Moreover, these CAM approaches present the advantage of potentially reducing associated side effects, making them valuable considerations in the holistic management of abdominal discomfort.

Therefore, the objective of the present invention is to address the limitations of conventional drugs by introducing a synergistic pharmaceutical composition that effectively healing abdominal pain and flatulence. This composition may be incorporated a varied combination of Chinese herbal ingredients as the active components, supplemented by the inclusion of additional excipients to optimize its therapeutic effects.

### SUMMARY OF THE INVENTION

Aspects of the present disclosure are to address the above-mentioned problems a disadvantages and to provide at least the advantages described throughout this patent specification.

Accordingly, an aspect of the present disclosure is to provide a pharmaceutical composition for healing abdominal pain and flatulence comprising Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita.

Another aspect of the present disclosure is to provide the pharmaceutical composition, wherein
said Poria cocos is about 4.2% by weight relative to total weight of said pharmaceutical composition;
said Atractylodes macrocephala is about 4.2% by weight relative to total weight of said pharmaceutical composition;
said Paeonia lactiflora is about 4.2% by weight relative to total weight of said pharmaceutical composition;
said Pseudostellaria heterophylla is about 2.5% by weight relative to total weight of said pharmaceutical composition;
said Pogostemon cablin is about 4.2% by weight relative to total weight of said pharmaceutical composition;
said Citrus reticulata is about 5.6% by weight relative to total weight of said pharmaceutical composition;
said Amomum villosum is about 7.3% by weight relative to total weight of said pharmaceutical composition;
said Evodia rutaecarpa is about 4.2% by weight relative to total weight of said pharmaceutical composition;
said Alpinia officinarum is about 3.1% by weight relative to total weight of said pharmaceutical composition;
said Zingiber officinale is about 2.5% by weight relative to total weight of said pharmaceutical composition;
said Astragalus membranaceus is about 4.2% by weight relative to total weight of said pharmaceutical composition;
said Dioscorea opposite is about 4.2% by weight relative to total weight of said pharmaceutical composition;
said Angelica dahurica is about 4.2% by weight relative to total weight of said pharmaceutical composition;
said Panax ginseng is about 3.1% by weight relative to total weight of said pharmaceutical composition;
said Corydalis yanhusuo is about 4.2% by weight relative to total weight of said pharmaceutical composition;
said Glycyrrhiza uralensis is about 2.5% by weight relative to total weight of said pharmaceutical composition;
said Cyperus rotundus is about 3.1% by weight relative to total weight of said pharmaceutical composition;
said Hordeum vulgare is about 4.2% by weight relative to total weight of said pharmaceutical composition;
said Lindera aggregate is about 4.2% by weight relative to total weight of said pharmaceutical composition;
said Melia toosendan is about 3.1% by weight relative to total weight of said pharmaceutical composition;
said Pinellia ternate is about 3.1% by weight relative to total weight of said pharmaceutical composition;
said Foeniculum vulgare is about 2.5% by weight relative to total weight of said pharmaceutical composition;
said Elettaria cardamomum is about 2.1% by weight relative to total weight of said pharmaceutical composition;
said Curcuma longa is about 3.1% by weight relative to total weight of said pharmaceutical composition;
said Atractylodes lancea is about 4.2% by weight relative to total weight of said pharmaceutical composition;
said Triticum aestivum is about 6.2% by weight relative to total weight of said pharmaceutical composition;
said Mentha x piperita is about 0.4% by weight relative to total weight of said pharmaceutical composition.

Another aspect of the present disclosure is to provide the pharmaceutical composition, wherein said pharmaceutical composition further comprising a sweetener.

Another aspect of the present disclosure is to provide the pharmaceutical composition, wherein said sweetener is sucralose.

Another aspect of the present disclosure is to provide the pharmaceutical composition, wherein said pharmaceutical composition further comprising a preservative.

Another aspect of the present disclosure is to provide the pharmaceutical composition, wherein said preservative is sodium benzoate.

Another aspect of the present disclosure is to provide the pharmaceutical composition, wherein said pharmaceutical composition is in the form of a cream, a lotion, or a gel.

Another aspect of the present disclosure is to provide the pharmaceutical composition, wherein said pharmaceutical composition is in the form of a liquid, a powder, a capsule, or a tablet.

Another aspect of the present disclosure is to provide the use of the pharmaceutical composition in the preparation of a medicament for use in the treatment of abdominal pain and flatulence by topical administration.

Another aspect of the present disclosure is to provide the use of the pharmaceutical composition in the preparation of a medicament for use in the treatment of abdominal pain and flatulence by oral administration.

### DETAILED DESCRIPTION

While embodiments of this invention can take many different forms, the preferred embodiments will be described herein in detail with the understanding that the present invention is to be considered as an exemplification of the principles of the invention, as well as the best mode of practicing same, and is not intended to limit the invention to the specific embodiment illustrated.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purposes only and not for the purpose of limiting the present disclosure as defined by the appended claims and their equivalents. Depending on the context of the sentence, the reading of this patent specification should be done with the same concept as “reject the absurd”, wherein if possible, a specification should be construed so as not to lead to a foolish result or one which the patentee could not have contemplated.

The term “about” as used herein, the particular value as determined by one skilled in the art (values depend on the manner in which is measured or determined), an acceptable error range, that is, the measuring system means that therein within the limits. For example, “about” can mean within standard deviations above the 1 or 1 per practice in the art. If a specific value is set forth in the application and claims, unless otherwise stated, the term “about” means within an acceptable error range for the particular value. If rigidity is needed in interpreting this term, in this application, the term about may be suggested as ±5% of the given value or can be considered as a range of effective value.

The term “qi” (pronounced “chee”) as used herein, represents the vital energy that circulates within the body. This energy's movement is known as qi flow, and it plays a significant role in TCM practices. Meridians, regarded as pathways or channels, are believed to facilitate the flow of qi throughout the body. These meridians serve as connections linking organs, muscles, and tissues to various areas of the body. When qi flows smoothly through these meridians, it is believed to enhance overall health and well-being. However, disruptions in qi flow can lead to imbalances and manifest in various health conditions. For example, blockages in meridians can cause qi stagnation, resulting in discomfort, inflammation, or other symptoms.

The term “Yang” as used herein, refers to one of the fundamental energetic forces within the body. Yin energy is responsible for maintaining moisture, lubrication, and substance in the body. It is associated with the organs and tissues that store, nourish, and provide structure, such as the liver, kidneys, lungs, and blood. Yin energy is also associated with emotions such as tranquility, introspection, and contentment. When there is an imbalance or deficiency in Yin energy, it can result in symptoms such as dryness, thirst, hot flashes, restlessness, insomnia, and excessive heat. This imbalance may also affect the quality of body fluids, leading to dry skin, brittle hair, and weak joints.

As used in this specification and the appended claims, the singular forms “a”, “an”, and “the” encompass embodiments having plural referents, unless the content clearly dictates otherwise. As used in this specification, the use of a singular form of a term can encompass embodiments including more than one of such terms, unless the context clearly dictates otherwise. As used in this specification and the appended claims, the term “or” is generally employed in its sense including “either or both” unless the context clearly dictates otherwise.

The present invention relates to a composition, specifically a pharmaceutical composition designed to alleviate abdominal pain and flatulence. This composition comprises a combination of herbal components, which serve as active ingredients. The herbal components in the composition are Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita. In a preferred embodiment, the herbal components of the composition are present in the proportion of about 4.2% by weight of Poria cocos; about 4.2% by weight of Atractylodes macrocephala; about 4.2% by weight of Paeonia lactiflora; about 2.5% by weight of Pseudostellaria heterophylla; about 4.2% by weight of Pogostemon cablin; about 5.6% by weight of Citrus reticulata; about 7.3% by weight of Amomum villosum; about 4.2% by weight of Evodia rutaecarpa; about 3.1% by weight of Alpinia officinarum; about 2.5% by weight of Zingiber officinale; about 4.2% by weight of Astragalus membranaceus; about 4.2% by weight of Dioscorea opposite; about 4.2% by weight of Angelica dahurica; about 3.1% by weight of Panax ginseng; about 4.2% by weight of Corydalis yanhusuo; about 2.5% by weight of Glycyrrhiza uralensis; about 3.1% by weight of Cyperus rotundus; about 4.2% by weight of Hordeum vulgare; about 4.2% by weight of Lindera aggregate; about 3.1% by weight of Melia toosendan; about 3.1% by weight of Pinellia ternate; about 2.5% by weight of Foeniculum vulgare; about 2.1% by weight of Elettaria cardamomum; about 3.1% by weight of Curcuma longa; about 4.2% by weight of Atractylodes lancea; about 6.2% by weight of Triticum aestivum; and about 0.4% by weight of Mentha x piperita. Weight fractions are relative to the total weight of the composition. The proportions of each herbal component of the composition of the present invention are displayed in the following Table 1.

TABLE 1
Proportions of each herbal component in
the composition of the present invention

| Active Ingredients | Weight (mg) | Percentage (%) |
|---|---|---|
| Poria cocos | 20 | 4.2 |
| Atractylodes macrocephala | 20 | 4.2 |
| Paeonia lactiflora | 20 | 4.2 |
| Pseudostellaria heterophylla | 12 | 2.5 |
| Pogostemon cablin | 20 | 4.2 |
| Citrus reticulata | 27 | 5.6 |
| Amomum villosum | 35 | 7.3 |
| Evodia rutaecarpa | 20 | 4.2 |
| Alpinia officinarum | 15 | 3.1 |
| Zingiber officinale | 12 | 2.5 |
| Astragalus membranaceus | 20 | 4.2 |
| Dioscorea opposite | 20 | 4.2 |
| Angelica dahurica | 20 | 4.2 |
| Panax ginseng | 15 | 3.1 |
| Corydalis yanhusuo | 20 | 4.2 |
| Glycyrrhiza uralensis | 12 | 2.5 |
| Cyperus rotundus | 15 | 3.1 |
| Hordeum vulgare | 20 | 4.2 |
| Lindera aggregate | 20 | 4.2 |
| Melia toosendan | 15 | 3.1 |
| Pinellia ternate | 15 | 3.1 |
| Foeniculum vulgare | 12 | 2.5 |
| Elettaria cardamomum | 10 | 2.1 |
| Curcuma longa | 15 | 3.1 |
| Atractylodes lancea | 20 | 4.2 |
| Triticum aestivum | 30 | 6.2 |
| Mentha × piperita | 2 | 0.4 |

In accordance with the present invention, the combination of the aforementioned herbal components as the active ingredients in the composition of the present invention plays a crucial role in effectively healing abdominal pain and flatulence, which commonly occur in various gastrointestinal conditions such as gastritis, gastroenteritis, and duodenal bulb ulcers. These conditions can lead to inflammation, irritation, or infection in the digestive system, resulting in the mentioned symptoms. Through synergistic effects, the carefully selected herbal components in the composition of the present invention work together to achieve the desired therapeutic outcomes. The selection of these herbal components is guided by the principles of Traditional Chinese Medicine (TCM) theory and clinical expertise, ensuring their effectiveness in treating these conditions.

The composition formulated by the present invention is designed to be directly absorbed by the body, resulting in fast-acting effects and a shortened treatment duration. This focused approach not only facilitates the healing of abdominal pain and flatulence but it also reduces the occurrence of ailments associated with diverse gastrointestinal conditions, promoting overall well-being. Moreover, the composition is free from side effects and poses no harm to the human body, effectively promoting overall well-being without any adverse consequences.

According to Traditional Chinese Medicine (TCM), the herbal components employed in the composition of the present invention have a significant historical significance in traditional remedies for a wide range of health conditions. Poria cocos, renowned for its sweet, light, and flat characteristics, is particularly valued for its diuretic and dampness-regulating properties, as well as its ability to strengthen the spleen and heart. Atractylodes macrocephala, with its bitter, sweet, and warm nature, is known for its efficacy in fortifying the spleen and qi, as well as its ability to address dampness and excessive sweating. Paeonia lactiflora, characterized by its bitter and sour taste, is recognized for its blood-tonifying effects, particularly on the liver, its analgesic properties, cooling effects on the blood, regulation of qi movement, and alleviation of spasms. Pseudostellaria heterophylla commonly known as “Prince Ginseng” or “False Starwort,” is a sweet-tasting herb that is often used for its potential to tonify qi, nourish the spleen, and promote overall vitality and well-being. Its traditional usage also includes supporting the immune system, enhancing digestion, and assisting in recovery from illness or fatigue. Pogostemon cablin is an aromatic herb with its earthy and musky fragrance and has soothing properties that can calm the mind, promote relaxation, and alleviate stress and anxiety. Additionally, Pogostemon cablin is believed to have antibacterial and anti-inflammatory effects, making it beneficial for skin care and wound healing. Citrus reticulata, a citrus fruit characterized by its sweet and tangy taste, is renowned for its ability to support digestion and alleviate symptoms like abdominal pain and flatulence. It is recognized for its digestive properties, making it beneficial for maintaining a healthy digestive system. The essential oil extracted from Citrus reticulata is widely utilized in aromatherapy to enhance mood, reduce stress levels, and induce a sense of relaxation and calmness. Amomum villosum, known for its unique smoky and earthy flavour, enhances the taste profile of various dishes, bringing depth and complexity to culinary creations. Within traditional medicine, Amomum villosum is highly regarded for its beneficial effects on digestion, providing relief from abdominal pain, bloating, and flatulence. It is believed to promote digestion, alleviate nausea, and contribute to overall digestive well-being. Furthermore, it is valued for its warming properties, which may have potential benefits in improving circulation and respiratory health. Evodia rutaecarpa known for its warming properties is frequently employed for its therapeutic benefits. In TCM, it is highly regarded for its ability to support digestion, alleviate abdominal pain and discomfort, and ease symptoms associated with gastrointestinal ailments like bloating and flatulence. Additionally, it is believed to have a positive influence on blood circulation, contributing to improved overall circulation and relief from related symptoms. Moreover, Evodia rutaecarpa is often utilized for its potential analgesic and anti-inflammatory properties, making it valuable for managing pain and inflammation in diverse conditions. Alpinia officinarum, with its unique aromatic flavour, is renowned for its digestive properties. In TCM, this herb is highly regarded for its ability to relieve symptoms like abdominal pain, bloating, and flatulence. It is believed to enhance digestion, boost appetite, and promote overall gastrointestinal well-being. Moreover, Alpinia officinarum is recognized for its potential anti-inflammatory and antioxidant effects, contributing to its positive impact on overall health and well-being. Zingiber officinale is characterized as having a hot nature and a bitter and pungent taste. According to TCM principles, Zingiber officinale has an affinity for the heart, spleen, stomach, lung, and large intestine organs. It is revered for its digestive properties, aiding in the relief of symptoms such as abdominal pain, bloating, and flatulence, while promoting optimal gastrointestinal health. It is also highly regarded for its analgesic properties, particularly in alleviating lower or middle-burner pain associated with cold conditions. Furthermore, Zingiber officinale is believed to have a positive influence on blood circulation, contributing to improved overall circulation and the alleviation of related symptoms. Its potential anti-inflammatory and antioxidant effects further enhance its health-promoting qualities. Astragalus membranaceus possesses a warm nature and a sweet taste. In line with TCM principles, Astragalus membranaceus is closely linked to the lung and spleen organs. It is believed to tonify Qi, the vital energy of the body, and strengthen the immune system. Astragalus membranaceus is often used to enhance overall vitality, support healthy digestion, and promote respiratory health. It is also recognized for its potential adaptogenic properties, helping the body adapt to stress and promoting general well-being. Dioscorea opposite is characterized by its cooling nature and sweet taste. According to TCM principles, Dioscorea opposita is associated with the spleen, lung, and kidney organs. It is believed to have nourishing properties, particularly in relation to the spleen and stomach, supporting healthy digestion and aiding in the tonification of Qi and Yin energies. Dioscorea opposita is often used to promote the production of body fluids, alleviate dryness, and strengthen the lungs. Additionally, it is recognized for its potential benefits in tonifying the kidneys and promoting overall vitality and well-being. Angelica dahurica is a pungent and warm herb known for its dispersing and drying properties. It has an affinity for the lung, stomach, and bladder organs. It is commonly used to relieve pain, reduce swelling, and treat conditions characterized by dampness and stagnation. Angelica dahurica is particularly effective in resolving external conditions such as headaches, nasal congestion, and sinusitis. It also exhibits antimicrobial properties and is beneficial for respiratory and skin health. Panax ginseng, with its warm nature and a taste that is both sweet and bitter, is closely linked to the lung and spleen organs. It is frequently employed to replenish Qi, boost both physical and mental stamina, and strengthen the immune system. Panax ginseng is highly regarded for its adaptogenic qualities, aiding the body in adapting to stress and promoting equilibrium. Moreover, it is commonly utilized to support cardiovascular health, enhance cognitive function, and promote overall wellness. Corydalis yanhusuo, known for its bitter taste, is associated with the liver, spleen, and heart. It is frequently employed to alleviate pain, especially menstrual cramps and abdominal pain. Its analgesic and anti-inflammatory properties make it effective in managing different types of pain. Moreover, Corydalis yanhusuo is recognized for its potential to induce sedation and promote a sense of calm, aiding in relaxation and stress relief. Glycyrrhiza uralensis, known for its sweet taste, is associated with the spleen, stomach, and lung organs. It is widely used for its medicinal properties, particularly in harmonizing and tonifying the body, especially the digestive system. It is believed to possess anti-inflammatory, expectorant, and detoxifying effects. Glycyrrhiza uralensis is commonly employed to soothe gastrointestinal problems, alleviate coughs and respiratory conditions, and enhance overall respiratory health. It is also highly valued for its ability to boost immune function and support hormonal balance. Cyperus rotundus is an herb with a warm and pungent nature and is associated with the spleen and liver organs. It is commonly used to improve digestion and alleviate symptoms such as abdominal pain, bloating, and flatulence. With its diuretic properties, it supports kidney function and helps address water retention. Cyperus rotundus also exhibits anti-inflammatory and analgesic effects, making it particularly useful for relieving menstrual cramps. This versatile herb is valued in TCM for its ability to promote digestive health, address water-related issues, and manage pain. Hordeum vulgare (“Barley”) is associated with the spleen, stomach, and liver organs. It has a neutral to warm temperature and a sweet taste with a touch of saltiness. Its primary actions involve harmonizing and tonifying the spleen and stomach, making it beneficial for conditions like food stagnation, epigastric pain, and loss of appetite. It can also alleviate lactation discomfort and regulate Liver qi, addressing symptoms such as epigastric distension and belching. Hordeum vulgare is valued for its ability to promote digestion, relieve food stagnation, and tonify the qi of the middle burner, improving overall gastrointestinal health. Lindera aggregate is a warm and pungent herb associated with the liver, spleen, and kidney organs. It is used to promote qi circulation, relieve pain, and improve digestion. Lindera aggregate has analgesic and anti-inflammatory effects, making it effective for various types of pain, including abdominal and menstrual pain. It also supports kidney function and urinary health. Melia toosendan, an herb known for its bitter and pungent taste, is closely linked to the liver and stomach organs. Its main applications in TCM include enhancing qi flow, alleviating pain, and addressing digestive problems. With its reputed antiparasitic and antimicrobial properties, Melia toosendan is effective against intestinal parasites and specific infections. Its anti-inflammatory effects further contribute to its pain-relieving abilities. Additionally, it is occasionally utilized for supporting liver health and managing certain skin conditions. Pinellia ternate is a pungent and warm herb associated with the lung, spleen, and stomach. It is primarily used to address respiratory and digestive issues. It is known for its ability to transform phlegm, alleviate cough, and promote the flow of qi. It is effective in treating conditions such as cough with phlegm, nausea, vomiting, and excessive sputum production.

Additionally, it helps regulate the middle burner and harmonize the stomach, making it beneficial for digestive disorders like indigestion and abdominal distension. Foeniculum vulgare is a herb with a sweet and aromatic taste. It is associated with the stomach and liver organs. It is widely used for its various medicinal properties, such as promoting digestion, relieving digestive discomforts such as bloating and flatulence, and supporting overall gastrointestinal health. It is also valued for its carminative effects, helping to ease colic and soothe the digestive system. Additionally, it is believed to have antimicrobial and anti-inflammatory properties, making it beneficial for addressing certain infections and reducing inflammation. Elettaria cardamomum is an aromatic herb with a distinct and refreshing taste. It is associated with the spleen and stomach. It is known to promote digestion, alleviate digestive discomforts such as bloating and indigestion, and support overall gastrointestinal health. It is also recognized for its warming properties, which can help relieve cold sensations in the body. Curcuma longa is an herb with a warm and bitter taste. It is associated with the spleen, stomach, and liver organs. It is highly valued for its medicinal properties, including its potent anti-inflammatory and antioxidant effects. It supports digestion, relieves digestive discomfort, and promotes gastrointestinal health. It also possesses immune-boosting properties and contributes to joint health. Atractylodes lancea has a bitter taste and warm nature, and it is associated with the spleen and stomach organs. Atractylodes lancea is primarily used for promoting digestion, relieving bloating, and improving appetite. It also has diuretic properties and may enhance the immune system and overall vitality. Triticum aestivum, commonly known as wheat, is a cereal grain abundant in carbohydrates, dietary fiber, and vital nutrients like vitamins and minerals. It plays a fundamental role in both traditional and contemporary diets, supplying individuals with energy and nourishment. Mentha x piperita (“peppermint”), is valued for its cooling and dispersing properties. It is primarily used to clear heat, resolve exterior conditions, promote digestion, and soothe the liver. It is also commonly utilized to alleviate symptoms such as headaches, fever, sore throat, and digestive disorders. Additionally, it is also often used in TCM herbal formulas and as a flavouring agent in herbal teas and remedies.

The present invention also provides a pharmaceutical composition for use in the treatment of abdominal pain and flatulence through topical or oral administrations. For topical administration, the composition is specifically formulated for direct application to the skin or affected area. It may comprise various components, including active ingredients, excipients, and delivery agents, which work synergistically to enhance the penetration of the active ingredients through the skin. The composition may be prepared as creams, ointments, gels, lotions, or patches, depending on the desired application and the specific properties of the active ingredients. According to the invention, creams, lotions, or gels are the preferred forms of the composition of the present invention for topical administration. For topical administration, the dosage of the composition may vary depending on the condition being treated. The recommended daily dosage will be determined by the selected formulation. For instance, it may be recommended to apply the composition once or twice daily, depending on the severity of the condition and the instructions provided.

For oral administration, the composition is specifically designed to be taken by mouth and may be available in the form of tablets, capsules, liquids, or suspensions. This oral formulation aims to effectively deliver the active ingredients to their intended site of action, which can encompass the gastrointestinal tract, bloodstream, or specific organs. According to the invention, liquids, powders, capsules, or tablets are the preferred forms of the composition of the present invention for oral administration. The liquid formulation described herein is conveniently packaged in sachets for easy use. On the other hand, the powder formulation is enclosed within a tea bag, allowing it to be prepared as a decoction through steeping or boiling in water. Additionally, the powder formulation can be enclosed within soft or hard gelatin capsules, creating a capsule form for administration. As for the tablet, it can be a bilayer or trilayer tablet with an outer protective coating or finishing layer. The outer protective coating, applied over the bilayer or trilayer tablets, may consist of conventional coating formulations containing film-formers or binders such as hydroxypropyl methylcellulose (HPMC), ethyl cellulose, cellulose acetate, polyvinyl alcohol-maleic anhydride copolymers, acrylic copolymers, beta-pinene polymers, and glyceryl esters of wood resins. Plasticizers like polyethylene glycol, triethyl citrate, diethyl phthalate, propylene glycol, glycerin, and butyl phthalate can also be included. The film-formers are applied using solvent systems containing solvents such as water, alcohols (methyl, ethyl, or isopropyl), ketones (acetone or ethyl methyl ketone), and chlorinated hydrocarbons (methylene chloride, dichloroethane, or 1,1,1-trichloroethane). For oral administration, the dosage of the composition may vary depending on the condition being treated. The recommended daily dosage will depend on the chosen formulation. For example, it may be advised to take one dosage of the composition 2 to 3 times per day.

The composition may incorporate a pharmaceutically acceptable carrier, which is essential for facilitating the application or dispersion of the active ingredient to the targeted site. This carrier also allows for the safe storage, transport, and handling of the composition while maintaining its efficacy. Pharmaceutical carriers that are suitable for use in the composition and formulation thereof are well-known to those skilled in the art. There are no particular restrictions on the selection of carriers within the scope of this invention. Additionally, the composition may include additives, such as wetting agents, dispersing agents, stickers, adhesives, emulsifying agents, solvents, coatings, antibacterial and antifungal agents (e.g., phenol, sorbic acid, chlorobutanol), isotonic agents (e.g., sugars or sodium chloride), preservatives (e.g., sodium benzoate), and other components commonly employed in pharmaceutical practice. The additives and carriers are selected such that they do not cause permanent damage to mammals, including humans. According to the invention, the composition, intended for both oral and topical administration, incorporates a preservative, preferably sodium benzoate. Sodium benzoate effectively serves as a preservative in both oral and topical formulations, preventing the growth of microorganisms and ensuring the stability of the composition throughout its shelf life. In the oral formulation, sodium benzoate acts as an inhibitor, preventing the growth of bacteria, yeast, and mold that could contaminate the product and maintain its integrity and quality. Similarly, in topical formulations, sodium benzoate inhibits the growth of bacteria, yeast, and mold, preserving the efficacy and quality of the cream, lotion, or gel. The concentration of sodium benzoate used as a preservative in both oral and topical formulations should comply with regulatory guidelines and fall within suitable limits to guarantee safety and tolerance for both oral and topical administration.

The composition of the present invention can incorporate various pharmaceutically acceptable excipients. In this context, an excipient refers to a non-therapeutic agent that acts as a carrier or vehicle to transport therapeutic agents or enhance the properties of a pharmaceutical composition, such as storage, handling, or dose-forming properties. Excipients encompass a wide range of substances, including diluents, disintegrants, binding agents, adhesives, wetting agents, lubricants, glidants, surface modifiers, flavorings, fragrances, dyes, and other additives that improve appearance or mask unpleasant tastes or odors. These excipients can be used in solid, semi-solid, liquid, or combined states, depending on the desired dosage form. The preparation of compositions with excipients can be accomplished through various pharmacy techniques involving mixing the excipient with a therapeutic agent or drug. For example, in liquid formulations, water can be employed as an excipient. Additionally, the composition of the liquid formulation may be incorporated with one or more sweeteners as excipients to improve taste and make it more palatable. Various sweeteners can be used, such as aspartame, cyclamate, dextrose, isomaltose, maltitol, mannitol, saccharin, sorbitol, stevia, xylitol, or similar substances. It is recommended that the liquid formulation of the composition of the present invention contains sweeteners chosen from the group consisting of sucralose for optimal sweetness. As for the oral formulation such as powders, capsules, or tablets, the composition may also be incorporated with one or more binding agents or adhesives as excipients. These binding agents and adhesives play a crucial role in providing cohesiveness to the powder during tablet compression, allowing for normal processing operations like sizing, lubrication, compression, and packaging, while ensuring the tablet disintegrates upon ingestion for efficient absorption. Suitable binding agents and adhesives include sucrose, gelatin, glucose, starch, or similar substances, either individually or in combination.

For topical formulations, the selection and incorporation of excipients play a crucial role in ensuring desired properties and efficacy. Excipients commonly used in topical formulations may include but are not limited to emulsifiers, thickeners, humectants, penetration enhancers, preservatives, antioxidants, and fragrance agents. These excipients contribute to the stability, texture, spread ability, absorption, and overall sensory experience of the topical composition. Emulsifiers are used to stabilize the mixture of oil and water phases in emulsion-based formulations like creams or lotions. Thickeners enhance the viscosity and provide the desired consistency of the formulation. Humectants help to retain moisture and prevent drying of the skin. Penetration enhancers aid in improving the absorption of active ingredients into the skin. Preservatives ensure the microbiological safety and shelf life of the formulation. Antioxidants help to protect the formulation from oxidative degradation. Fragrance agents contribute to the sensory appeal of the topical product. The selection and inclusion of these excipients in the composition for a topical application depend on the specific requirements and desired characteristics of the formulation. By utilizing appropriate excipients in the preparation of lotions, creams, or gels, the composition can be optimized for effective and pleasant topical administration, providing the desired therapeutic benefits.

The present invention can be prepared using conventional techniques widely employed in the pharmaceutical industry. Various embodiments of the invention may be employed for the preparation of the composition, such as dry mixing, direct compression, milling, dry or non-aqueous granulation, wet granulation, or fusion methods used for producing solid dosage forms. The following examples describe a method for preparing the composition of the present invention.

### Example 1

### A Method of Preparing a Pharmaceutical Composition of the Present Invention in a Liquid Form

The method of preparing a pharmaceutical composition of the present invention in a liquid form was carried out in a closed system to ensure the accuracy and quality of the finished result. The process began with the precise weighing of the herbal components that served as active ingredients [i.e., Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita]. Herein, the herbal components of the composition were present in the proportion of about 4.2% by weight of Poria cocos; about 4.2% by weight of Atractylodes macrocephala; about 4.2% by weight of Paeonia lactiflora; about 2.5% by weight of Pseudostellaria heterophylla; about 4.2% by weight of Pogostemon cablin; about 5.6% by weight of Citrus reticulata; about 7.3% by weight of Amomum villosum; about 4.2% by weight of Evodia rutaecarpa; about 3.1% by weight of Alpinia officinarum; about 2.5% by weight of Zingiber officinale; about 4.2% by weight of Astragalus membranaceus; about 4.2% by weight of Dioscorea opposite; about 4.2% by weight of Angelica dahurica; about 3.1% by weight of Panax ginseng; about 4.2% by weight of Corydalis yanhusuo; about 2.5% by weight of Glycyrrhiza uralensis; about 3.1% by weight of Cyperus rotundus; about 4.2% by weight of Hordeum vulgare; about 4.2% by weight of Lindera aggregate; about 3.1% by weight of Melia toosendan; about 3.1% by weight of Pinellia ternate; about 2.5% by weight of Foeniculum vulgare; about 2.1% by weight of Elettaria cardamomum; about 3.1% by weight of Curcuma longa; about 4.2% by weight of Atractylodes lancea; about 6.2% by weight of Triticum aestivum; and about 0.4% by weight of Mentha x piperita. The weight fractions were relative to the total weight of the composition. Then, distilled water was also measured separately.

A high-shear dispersion homogenous mixing method was then used to blend these ingredients. To create a consistent and homogeneous mixture, this process made use of specialized machinery like high-shear mixers. To keep the correct conditions, the mixing procedure was conducted at a controlled temperature of 15° C.

The mixture then underwent a critical process known as light emission LASER vacuum sterilization after being well mixed to produce a pharmaceutical composition in a liquid form. This procedure utilized the latest technology in the field of sterilization to get rid of any potential bacteria or pollutants in the combination, guaranteeing the sterility of the finished product. This sterilization process was essential for ensuring the safety and effectiveness of the pharmaceutical composition.

The pharmaceutical composition in the liquid form then moved on to the precision filling procedure after being sterilized. The sterilized liquid formulation of the pharmaceutical composition was carefully transferred under controlled pressure into the proper containers, such as sachets or bottles. To ensure precise dosing and reduce the possibility of introducing contaminants that can lower the quality of the final product, the pressurized filling procedure was crucial. The filled containers were then put through a packing process that involves labelling, sealing, and other essential procedures.

### Example 2

### A Method of Preparing a Pharmaceutical Composition of the Present Invention in a Powder Form Enclosed in a Sachet

The method of preparing a pharmaceutical composition of the present invention in powder form enclosed in a sachet involved a series of essential steps to ensure the quality and efficacy of the final product.

To ensure the proper proportions, the method started with the careful weighing of the necessary ingredients which were herbal components that served as active ingredients [i.e., Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita]. Herein, the herbal components of the composition were present in the proportion of about 4.2% by weight of Poria cocos; about 4.2% by weight of Atractylodes macrocephala; about 4.2% by weight of Paeonia lactiflora; about 2.5% by weight of Pseudostellaria heterophylla; about 4.2% by weight of Pogostemon cablin; about 5.6% by weight of Citrus reticulata; about 7.3% by weight of Amomum villosum; about 4.2% by weight of Evodia rutaecarpa; about 3.1% by weight of Alpinia officinarum; about 2.5% by weight of Zingiber officinale; about 4.2% by weight of Astragalus membranaceus; about 4.2% by weight of Dioscorea opposite; about 4.2% by weight of Angelica dahurica; about 3.1% by weight of Panax ginseng; about 4.2% by weight of Corydalis yanhusuo; about 2.5% by weight of Glycyrrhiza uralensis; about 3.1% by weight of Cyperus rotundus; about 4.2% by weight of Hordeum vulgare; about 4.2% by weight of Lindera aggregate; about 3.1% by weight of Melia toosendan; about 3.1% by weight of Pinellia ternate; about 2.5% by weight of Foeniculum vulgare; about 2.1% by weight of Elettaria cardamomum; about 3.1% by weight of Curcuma longa; about 4.2% by weight of Atractylodes lancea; about 6.2% by weight of Triticum aestivum; and about 0.4% by weight of Mentha x piperita. The weight fractions were relative to the total weight of the composition.

The weighing components were then grounded to powder form and thoroughly mixed to ensure a uniform and homogeneous mixture. Subsequently, the mixture underwent a sterilization process, to effectively eliminate any potential microorganisms or contaminants. This process was essential for preserving the safety of the powder composition. Finally, the sterilized powder composition was subjected to a precision filling process, where it was accurately filled into sachets. The precision filling process ensured accurate dosing and consistent product quality. The sachets were then ready for the packaging process, which included appropriate labelling and sealing, preparing the pharmaceutical composition for distribution and use.

While this invention has been described by reference to certain specific embodiments and examples, it will be understood that this invention is capable of further modifications. This application is, therefore, intended to cover any variations, uses, or adaptations of the invention following the general principles thereof, and including such departures from the present disclosure as come within the known or customary practice in the art to which this invention pertains and falls within the limits of the appended claims.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

1. A pharmaceutical composition for healing abdominal pain and flatulence comprising Poria cocos, Atractylodes macrocephala, Paeonia lactiflora, Pseudostellaria heterophylla, Pogostemon cablin, Citrus reticulata, Amomum villosum, Evodia rutaecarpa, Alpinia officinarum, Zingiber officinale, Astragalus membranaceus, Dioscorea opposite, Angelica dahurica, Panax ginseng, Corydalis yanhusuo, Glycyrrhiza uralensis, Cyperus rotundus, Hordeum vulgare, Lindera aggregate, Melia toosendan, Pinellia ternate, Foeniculum vulgare, Elettaria cardamomum, Curcuma longa, Atractylodes lancea, Triticum aestivum, and Mentha x piperita.

2. The pharmaceutical composition as claimed in claim 1, wherein
said Poria cocos is about 4.2% by weight relative to total weight of said pharmaceutical composition;
said Atractylodes macrocephala is about 4.2% by weight relative to total weight of said pharmaceutical composition;
said Paeonia lactiflora is about 4.2% by weight relative to total weight of said pharmaceutical composition;
said Pseudostellaria heterophylla is about 2.5% by weight relative to total weight of said pharmaceutical composition;
said Pogostemon cablin is about 4.2% by weight relative to total weight of said pharmaceutical composition;
said Citrus reticulata is about 5.6% by weight relative to total weight of said pharmaceutical composition;
said Amomum villosum is about 7.3% by weight relative to total weight of said pharmaceutical composition;
said Evodia rutaecarpa is about 4.2% by weight relative to total weight of said pharmaceutical composition;
said Alpinia officinarum is about 3.1% by weight relative to total weight of said pharmaceutical composition;
said Zingiber officinale is about 2.5% by weight relative to total weight of said pharmaceutical composition;
said Astragalus membranaceus is about 4.2% by weight relative to total weight of said pharmaceutical composition;
said Dioscorea opposite is about 4.2% by weight relative to total weight of said pharmaceutical composition;
said Angelica dahurica is about 4.2% by weight relative to total weight of said pharmaceutical composition;
said Panax ginseng is about 3.1% by weight relative to total weight of said pharmaceutical composition;
said Corydalis yanhusuo is about 4.2% by weight relative to total weight of said pharmaceutical composition;
said Glycyrrhiza uralensis is about 2.5% by weight relative to total weight of said pharmaceutical composition;
said Cyperus rotundus is about 3.1% by weight relative to total weight of said pharmaceutical composition;
said Hordeum vulgare is about 4.2% by weight relative to total weight of said pharmaceutical composition;
said Lindera aggregate is about 4.2% by weight relative to total weight of said pharmaceutical composition;
said Melia toosendan is about 3.1% by weight relative to total weight of said pharmaceutical composition;
said Pinellia ternate is about 3.1% by weight relative to total weight of said pharmaceutical composition;
said Foeniculum vulgare is about 2.5% by weight relative to total weight of said pharmaceutical composition;
said Elettaria cardamomum is about 2.1% by weight relative to total weight of said pharmaceutical composition;
said Curcuma longa is about 3.1% by weight relative to total weight of said pharmaceutical composition;
said Atractylodes lancea is about 4.2% by weight relative to total weight of said pharmaceutical composition;
said Triticum aestivum is about 6.2% by weight relative to total weight of said pharmaceutical composition;
said Mentha x piperita is about 0.4% by weight relative to total weight of said pharmaceutical composition.

3. The pharmaceutical composition as claimed in claim 1, wherein said pharmaceutical composition further comprising a sweetener.

4. The pharmaceutical composition as claimed in claim 3, wherein said sweetener is sucralose.

5. The pharmaceutical composition as claimed in claim 1, wherein said pharmaceutical composition further comprising a preservative.

6. The pharmaceutical composition as claimed in claim 5, wherein said preservative is sodium benzoate.

7. The pharmaceutical composition for healing abdominal pain and flatulence as claimed in claim 1, wherein said pharmaceutical composition is in the form of a cream, a lotion, or a gel.

8. The pharmaceutical composition for healing abdominal pain and flatulence as claimed in claim 1, wherein said pharmaceutical composition is in the form of a liquid, a powder, a capsule, or a tablet.

9. Use of the pharmaceutical composition as claimed in claim 7 in the preparation of a medicament for use in the treatment of abdominal pain and flatulence by topical administration.

10. Use of the pharmaceutical composition as claimed in claim 8 in the preparation of a medicament for use in the treatment of abdominal pain and flatulence by oral administration.
